(19) United States
(12) Patent Application Publication
(10) Pub. No.: US 2002/0168326 A1
(43) Pub. Date: Nov. 14, 2002

(54) Hair conditioner and scalp treatment solution

(76) Inventor: John Lore, White Plains, MD (US)

Correspondence Address: John L. Welsh, WELSH & FLAXMAN LLC, Suite 112, 2341 Jefferson Davis Hwy., Arlington, VA, 22202, US

(21) Appl. No.: 09/850,071

(22) Filed: May 8, 2001

(51) Int. Cl. A61K 7/06

(52) U.S. Cl. 424/70.1

(57) ABSTRACT

A hair conditioner-scalp stimulator solution made of liquid extracts from coconut, lemon and lime fruit.

### BACKGROUND OF THE INVENTION

The present invention relates to a hair treatment product and more particular to a hair conditioner and/or scalp stimulant.

Various natural ingredients have been used as hair conditioners and the like as evidenced by the following prior art. U.S. Pat. No. 41,791 to Smith used lemon juice, castor oil, alcohol, oil of rose, water, burdock root and balsam as a compound for the hair. U.S. Pat. No. 50,901 to Carr describes a hair restorative made with juice of onion, alcohol and oils of rosemary, bergamot and lemon.

U.S. Pat. No. 75,806 to Sterns discloses a hair dressing compound formed of coconut fat and castor oil mixed in alcohol. U.S. Pat. No. 116,562 to Calabro discloses a hair remedy formed of lemon juice, olive oil and powdered sulfur. U.S. Pat. No. 130,720 to Johnson describes a hair restorative made from the ashes of willow, ashes of leather, coconut butter, tallow and white grape wine.

U.S. Pat. No. 1,378,731 to Silvera describes a hair restorer using coconut oil. U.S. Pat. No. 3,809,747 to Tuscano discloses a hair and scalp treatment using chili pepper and lemon. U.S. Pat. No. 3,824,304 to Villanueva is directed to a hair conditioner using coconut oil, sweet orange, capsicum juice and tincture of iodine.

The present invention is directed to a hair conditioner and scalp stimulator solution formed of natural fruit extract products including undiluted lemon juice, lime juice and coconut milk, that is, the liquid found within the interior of the coconut nut. The ingredients are mixed together and are used by applying the mixture to the scalp, preferably on a regular basis, by rubbing the solution in the scalp and massaging the area with the fingers.

The hair conditioner scalp stimulator of the present invention has been found to control scalp itch and dandruff, lessens hair loss, provides thicker hair with more body and increased sheen. The solution also controls scalp oil and stimulates growth of dormant hair.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limited, but merely as the basis for the claims and as a basis for teaching one skilled in the art how to make and/or use the invention.

The present invention is a hair conditioner and scalp stimulant made of equal parts of coconut milk, lemon juice and lime juice combined to provide a liquid solution for application to the hair and scalp. The hair conditioner and scalp stimulant of the present invention is made from organic substances available from natural growing fruit trees. The solution mixture is generally clear with a slight citrus color and exhibits a pleasant smell. A first ingredient is the milk from the coconut, which is the water found inside the fruity part of the coconut nut and is derived from the coconut palm or “cocos nucifera”. The tree is common in various tropical regions. A second ingredient of the hair conditioner scalp stimulator of the present invention is the juice from the fruit of the lime or “citrus aurantifolia”. This fruit is grown on trees widely found throughout tropical and sub-tropical areas of the world. A third ingredient is the juice from the fruit of the lemon “citrus limon”, from another tree widely grown throughout the tropical and sub-tropical areas of the world.

### EXAMPLE

The hair conditioner-scalp stimulator composition is prepared by mixing equal parts of coconut milk, lemon juice and lime juice. The coconut milk is extracted from the interior of whole coconuts by drilling a hole or otherwise opening the coconut shell. The lemon and lime juice is extracted by squeezing the fruit. Unadulterated, equal portions of the liquid are mixed using a suitable mixing device to form the mixture.

The hair conditioner, scalp stimulator may be used at room temperature, or it may be refrigerated. The solution is applied directly to the scalp and hair using a dropper or similar applicator device. Preferably, only several drops are necessary and they are massaged into the scalp briskly for one or two minutes to ensure that the solution penetrates the scalp and hair. After the solution is applied it dries in a non-sticky manner and may be kept on the scalp overnight.

Alternately, oils of the coconut, lemon and lime may be mixed to create a hair conditioner, scalp solution in accordance with the invention. The oils are obtained by squeezing the coconut pulp and squeezing the skins of the lemons and limes in a press. Equal amounts are mixed to form the hair conditioner, scalp stimulator solution.

The above describes preferred embodiments of the present invention. It will be appreciated that various changes and modifications may be made in keeping within the spirit and scope of the invention as defined by the following claims.

1. A hair conditioner-scalp stimulator comprising portions of liquid extracts derived from coconuts, lemons and limes.

2. The hair conditioner-scalp stimulator of claim 1 wherein said liquid extracts are coconut milk, lemon juice and lime juice.

3. The hair conditioner-scalp stimulator of claim 1 wherein said liquid extracts are coconut oil, lemon oil and lime oil.

4. A hair conditioner-scalp stimulator solution comprising equal portions of coconut milk, lemon juice and lime juice.

5. A hair conditioner-scalp stimulator solution comprising equal portions of coconut oil, lemon oil and lime oil.
